(19) United States
(12) Patent Application Publication
(10) Pub. No.: US 2023/0214994 A1
(43) Pub. Date: Jul. 6, 2023

(54) MEDICAL IMAGE STUDY DIFFICULTY ESTIMATION

(71) Applicant: MERATIVE US L.P., Ann Arbor, MI (US)

(72) Inventors: Sun Young Park, San Diego, CA (US); Dustin Michael Sargent, San Diego, CA (US); Benedikt Graf, Charlestown, MA (US); Larissa Christina Schudlo, Cambridge, MA (US); Marwan Sati, Mississauga (CA)

(21) Appl. No.: 17/569,465

(22) Filed: Jan. 5, 2022

(51) Int. Cl. G06T 7/00; G16H 50/20; G06F 40/20; G16H 30/40

(52) U.S. Cl. CPC G06T 7/0012; G16H 50/20; G06F 40/20; G16H 30/40; G06T 2207/20081

(57) ABSTRACT

Methods and systems for assigning a medical image study for review. One method includes receiving a plurality of labeled medical image studies and one or more prior image studies of a patient associated with each of plurality of labeled medical image studies. The method also includes creating a set of training data including the plurality of labeled medical image studies and the one or more prior image studies received for each of the plurality of labeled medical image studies and training an artificial intelligence (AI) system using the set of training data. In addition, the method includes estimating, using the AI system as trained, a difficulty metric for an unlabeled medical image study based on the unlabeled medical image study and one or more prior image studies of a patient associated with the unlabeled image study and assigning the unlabeled medical image study for review based on the difficulty metric.

### FIELD

Embodiments described herein relate to systems and methods for estimating a difficulty metric of a medical image study. Some systems and methods use various machine learning models of an artificial intelligence (AI) system to estimate a difficulty metric of a medical image study, wherein the medical image study is assigned for review based on the difficulty metric.

### SUMMARY

A medical image study may include one or more medical images captured of a patient. An image study may also include information regarding the patient, image study information, order information, or a combination thereof. A healthcare provider, such as a radiologist, may receive the medical image study for review and generation of an associated report (e.g., with annotations, notes, finding, diagnoses, etc.).

Difficulty varies amongst medical image studies depending on the content of the information of the medical image study and other related information. Relative Value Units (RVUs) are a current measure for standardizing a difficulty level of various types of medical imaging studies. RVUs may be used to determine reimbursement for healthcare providers for different study types. However, embodiments described herein recognize that RVUs do not account for many factors that significantly affect the complexity and difficulty of a medical image study, such as studies with current and multiple priors, clinical findings, demographic information of a patient (e.g., age, gender, body mass index, etc.), study details (contrast or no contrast), etc. Additionally, embodiments described herein recognize that greater amounts of relevant priors increase the amount of work needed to review the medical image study, particularly when the priors include multiple findings or impressions that may be correlated with artificial intelligence and computer aided diagnosis findings in a current exam. Embodiments described herein also recognize that accurately measuring study difficulty or complexity is important for efficient workload balancing and medical image study distribution among healthcare providers.

Accordingly, embodiments described herein provide methods and systems for estimating a difficulty metric of a medical image study. The methods and systems can use models of an artificial intelligence (AI) system to learn patterns of study difficulty using factors of information of the medical image study, for example, such as medical image study information, information regarding a patient, information regarding a prior image study, etc. In particular, embodiments described herein can use ensemble methods to account for factors of information of the medical image study that RVUs do not consider. Ensemble methods are a machine learning technique that combines various machine learning models to produce a predictive performance that any of the various machine learning models alone cannot produce. In addition to removing the time required for manual workload distribution, using an AI system as described herein to assign medical image studies to healthcare providers provides a difficulty metric, which is more effective than RVUs, to estimate and balance workload of healthcare providers. Furthermore, as compared to simple rules-based assignment system, the machine learning model of the AI system as described herein can automatically adjust over time to changing parameters of medical image studies.

Accordingly, embodiments described herein use models of an artificial intelligence (AI) system to automatically assign medical image studies to a healthcare provider. For example, one embodiment provides a computer-implemented method for assigning a medical image study for review. The method includes receiving a plurality of labeled medical image studies, wherein each of the plurality of labeled medical image studies including a medical image study and a label representing a difficulty of the respective medical image study. The method also includes receiving, for each of the plurality of labeled medical image studies, one or more prior image studies of a patient associated with the respective labeled medical image study. The method further includes creating a set of training data including the plurality of labeled medical image studies and the one or more prior image studies received for each of the plurality of labeled medical image studies and training an artificial intelligence (AI) system using the set of training data. In addition, the method includes estimating, using the AI system as trained, a difficulty metric for an unlabeled medical image study based on the unlabeled medical image study and one or more prior image studies of a patient associated with the unlabeled image study and assigning the unlabeled medical image study for review based on the difficulty metric.

Another embodiment provides a system for assigning a medical image study for review. The system includes an electronic processor. The electronic processor is configured to receive a plurality of labeled medical image studies, wherein each of the plurality of labeled medical image studies including a medical image study and a label representing a difficulty of the respective medical image study. The electronic processor is also configured to receive, for each of the plurality of labeled medical image studies, one or more prior image studies of a patient associated with the respective labeled medical image study, create a set of training data including the plurality of labeled medical image studies and the one or more prior image studies received for each of the plurality of labeled medical image studies, and train an artificial intelligence (AI) system using the set of training data. The electronic processor is further configured to estimate, using the AI system as trained, a difficulty metric for an unlabeled medical image study based on the unlabeled medical image study and one or more prior image studies of a patient associated with the unlabeled image study and assign the unlabeled medical image study for review based on the difficulty metric.

Yet a further embodiment provides a non-transitory computer-readable medium storing instructions that, when executed by an electronic processor, perform a set of functions. The set of functions include receiving a plurality of labeled medical image studies, wherein each of the plurality of labeled medical image studies including a medical image study and a label representing a difficult of the respective medical image study, and receiving, for each of the plurality of labeled medical image studies, one or more prior image studies of a patient associated with the respective labeled medical image study. The set of functions further include creating a set of training data including the plurality of labeled medical image studies and the one or more prior image studies received for each of the plurality of labeled medical image studies, training an artificial intelligence (AI) system using the set of training data, estimating, using the AI system as trained, a difficulty metric for an unlabeled medical image study based on the unlabeled medical image study and one or more prior image studies of a patient associated with the unlabeled image study, and assigning the unlabeled medical image study for review based on the difficulty metric.

Other aspects of the invention will become apparent by consideration of the detailed description and accompanying drawings.

### BRIEF DESCRIPTION OF THE DRAWINGS

FIG. 1 schematically illustrates a medical study assignment system according to some embodiments.

FIG. 2 schematically illustrates assignment of medical image studies to individual care provider worklists according to some embodiments.

FIG. 3A illustrates a training workflow of a difficulty model according to some embodiments.

FIG. 3B illustrates a medical image study scoring workflow of a difficulty model according to some embodiments.

FIG. 3C illustrates a scoring workflow of a model of the difficulty model of FIG. 3B.

FIG. 4 is a flowchart illustrating a method performed by the medical study assignment system of FIG. 1.

### DETAILED DESCRIPTION

Before any embodiments are explained in detail, it is to be understood that the embodiments are not limited in their application to the details of construction and the arrangement of components set forth in the following description or illustrated in the following drawings. Other embodiments are capable of being practiced or of being carried out in various ways.

It should be understood that although certain drawings illustrate hardware and software located within particular devices, these depictions are for illustrative purposes only. In some embodiments, the illustrated components may be combined or divided into separate software, firmware and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting, and coupling. Further, “connected” and “coupled” are not restricted to physical or mechanical connections or couplings, and may include electrical connections or coupling, whether direct or indirect. Also, electronic communications and notifications may be performed using any known means including direct connections, wireless connections, etc.

A plurality of hardware and software-based devices, as well as a plurality of different structural components may be utilized to implement the embodiments. In addition, embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects of the embodiments may be implemented in software (e.g., stored on non-transitory computer-readable medium) executable by one or more processors. As such, it should be noted that a plurality of hardware and software-based devices, as well as a plurality of different structural components, may be utilized to implement the embodiments. For example, “mobile device,” “computing device,” and “server” as described in the specification may include one or more electronic processors, one or more memory modules including non-transitory computer-readable medium, one or more input/output interfaces, and various connections (e.g., a system bus) connecting the components.

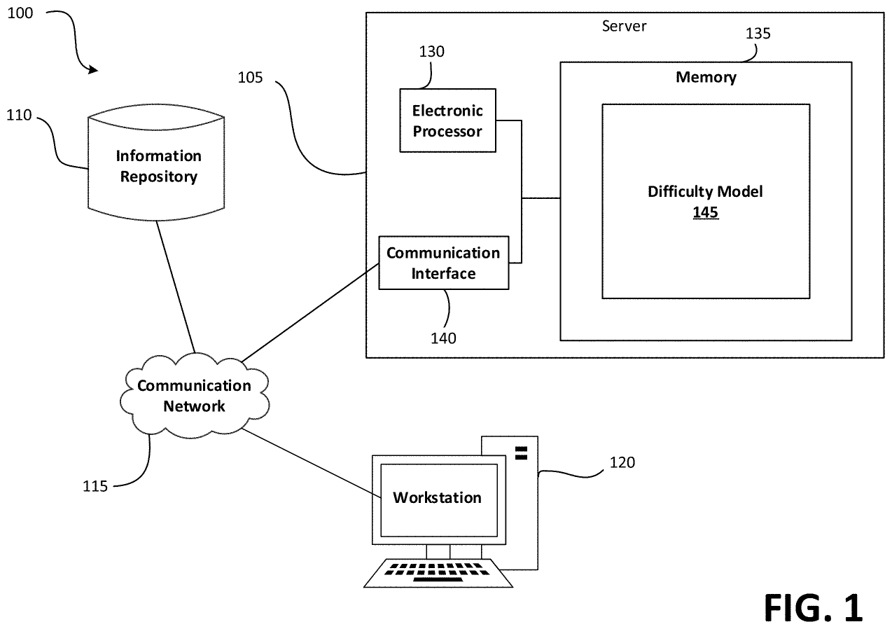

As described above, embodiments provided herein provide methods and systems for estimating a difficulty metric of a medical image study. FIG. 1 illustrates a medical image study assignment system 100 according to some embodiments. As illustrated in FIG. 1, the system 100 includes a server 105, an information repository 110, and a workstation 120. The server 105, the information repository 110, and the workstation 120 communicate over one or more wired or wireless communication networks 115. Portions of the wireless communication networks 115 may be implemented using a wide area network, such as the Internet, a local area network, such as a Bluetooth™ network or Wi-Fi, and combinations or derivatives thereof. It should be understood that the system 100 may include more or fewer servers and the single server 105 illustrated in FIG. 1 is purely for illustrative purposes. For example, in some embodiments, the functionality described herein is performed via a plurality of servers in a distributed or cloud-computing environment. Also, in some embodiments, the server 105 may communicate with multiple information repositories. Additionally, it should be understood that the system 100 may include more workstations and the single workstation 120 illustrated in FIG. 1 is purely for illustrative purposes. For example, in some embodiments, the system 100 includes a plurality of workstations 120, each workstation associated with a care provider. Also, in some embodiments, the components illustrated in system 100 may communicate through one or more intermediary devices (not shown).

The information repository 110 stores medical data, including, for example, medical image studies. A medical image study may comprise a plurality of images captured of a patient using an imaging modality. For example, the information repository 110 may include a picture archiving and communication system (PACS) that stores various types of medical images. In some embodiments, the information repository 110 may also store other medical data such as patient information, reports for prior exams, pathology reports or results, or the like. For example, in some embodiments, the information repository 110 may include an electronic medical record (EMR) system, hospital information system (HIS), a radiology information system (RIS). In some embodiments, the information repository 110 may also be included as part of the server 105. Also, in some embodiments, the information repository 110 may represent multiple servers or systems, such as for example, a PACS, an EMR system, a RIS, and the like. Accordingly, the server 105 may be configured to communicate with multiple systems or servers to perform the functionality described herein. Alternatively or in addition, the information repository 110 may represent an intermediary device configured to communicate with the server 105 and one or more additional systems or servers (e.g., a PACS, an EMR system, a RIS, etc.). Accordingly, the medical data stored in or accessible through the information repository 110 can include patient information, images, reports of findings, pathology reports or results, EMR information, historical reading times of the medical image studies, relative value units (RVUs), etc.

In some embodiments, the patient information stored in or accessible through the information repository 110 can include information such as demographic information, procedure history, disease history, etc. related to a specific patient.

The images stored in the information repository 110 are generated by an imaging modality (not shown), such as an X-ray, a computed tomography (CT) scanner, a magnetic resonance imaging (MRI) scanner, or the like. In some embodiments, the information repository 110 may also be included as part of an imaging modality. The images stored in the information repository 110 may be grouped into image studies. In some embodiments, images within an image study are generated by the same image modality (not shown) for a patient. In addition to one or more medical images, an image study can include metadata. The metadata may include study description, number of series/slices, an imaging modality type or identifier, and patient information. The metadata may be defined according to one or more standards for communicating medical data, such as, for example, the digital imaging and communications in medicine (DICOM) standard, the health level seven (HL7) standard, or the like.

Reports or findings stored in or accessible through the information repository 110 can include reports or findings automatically generated by one or more systems, such as, for example, one or more computer-aided diagnosis (CAD) systems, artificial intelligence systems, or the like. Alternatively or in addition, the reports and findings can include electronic reports or findings generated by a radiologist or other healthcare professional, such as for, example, an image study report, a pathology report, or the like. For example, a radiologist may use a RIS to create an electronic report for an image study, wherein the report includes findings or impressions, one or more diagnoses, annotations, measurements, or the like. Metadata regarding such reports or findings can also be stored in or accessible through the information repository 110. For example, timing information relating to completion of an image study report can be stored, which may represent how long it took a radiologist to read an image study and create the associated report. Similarly, other information relating to how a report was generated can be stored, such as, for example, what images (or what number of images) were reviewed as part of creating a report, what or what number of prior reports were reviewed as part of creating a report, or the like.

As illustrated in FIG. 1, the server 105 includes an electronic processor 130, a memory 135, and a communication interface 140. The electronic processor 130, the memory 135, and the communication interface 140 communicate wirelessly, over wired communication channels or buses, or a combination thereof. The server 105 may include additional components than those illustrated in FIG. 1 in various configurations. For example, in some embodiments, the server 105 includes multiple electronic processors, multiple memory modules, multiple communication interfaces, or a combination thereof. Also, it should be understood that the functionality described herein as being performed by the server 105 may be performed in a distributed nature by a plurality of computers located in various geographic locations. For example, the functionality described herein as being performed by the server 105 may be performed by a plurality of computers included in a cloud computing environment.

The electronic processor 130 may be, for example, a microprocessor, an application-specific integrated circuit (ASIC), and the like. The electronic processor 130 is generally configured to execute software instructions to perform a set of functions, including the functions described herein. The memory 135 includes a non-transitory computer-readable medium and stores data, including instructions executable by the electronic processor 130. The communication interface 140 may be, for example, a wired or wireless transceiver or port, for communication over the communication network 115 and, optionally, one or more additional communication networks or connections.

As illustrated in FIG. 1, the memory 135 of the server 105 includes a difficulty model 145, which may be part of a medical image study assignment engine executed via the server 105. The difficulty model 145 may be, for example, an artificial intelligence system. Additionally, the memory 135 may store a worklist table that identifies a workload of each of a plurality of healthcare providers working within the system 100, such as a plurality of radiologists. As medical image studies are generated or at a predetermined frequency (e.g., stored to the information repository 110), the server 105 uses the difficulty model 145 to determine a difficulty metric for each image study, wherein the difficult metric can be used to assign each the medical image study to a care provider (i.e., assign to a particular worklist table) within the system 100.

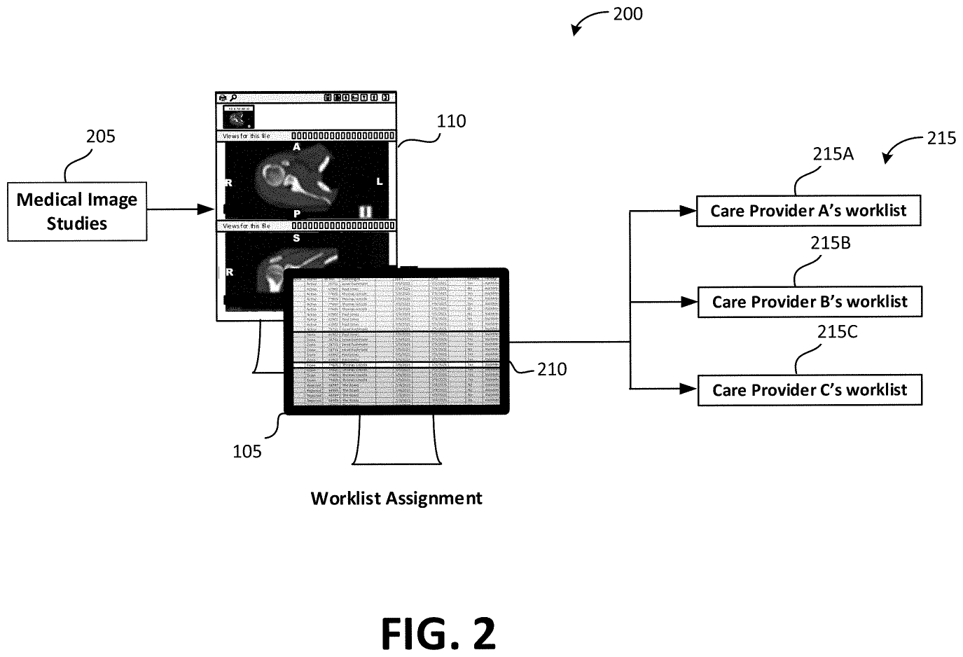

For example, FIG. 2 illustrates a workflow 200 for assigning medical image studies to care providers. As illustrated in FIG. 2, server 105 stores (or has access to) a worklist assignment table 210 that includes, among other things, an identifier for each medical image study needing review. The server 105 also stores (or has access to) a plurality of care provider worklists 215. As described in further detail below, the server 105 uses the difficulty model 145 to assign each medical image study (e.g., medical image study 205A) of the plurality of medical image studies 205 to one of the care provider worklists 215 (such as, for example, the care provider A worklist 215A, the care provider B worklist 215B, or the care provider C worklist 215C).

Server 105 receives completed medical image studies and associated medical information from the information repository 110 to train the difficulty model 145. The difficulty model 145 may be trained, for example, by a supervised learning method using labeled medical image studies to estimate a difficulty metric of a received unlabeled medical image study. A supervised learning method is a machine learning task that learns a function that maps an input to an output based on a set of input-output pairs. The set of input-output pairs (e.g., a set of training data) may include a medical image study (i.e., input) tagged with one or more labels and a difficulty metric (i.e., output). For example, an expert can provide a label (e.g., a numerical scale, such as from 1-10) for a medical image study that represents a difficulty of an image study. Alternatively or in addition, a label for a medical image study can be automatically assigned based on a predicted reading time associated with historical reading times of comparable medical image studies (e.g., excluding outliers).

The information used to train the difficulty model 145 (also referred to herein as the “training data”) can include a plurality completed or labeled (i.e., reviewed and assigned a difficulty label or score) image studies (images included in each study) and associated additional information. For example, the training data used to train the difficulty model 145 can include a plurality of factors that impact study difficulty and, thus, result in a more accurate model 145 that considers multiple factors that can influence the difficulty of a medical image study (i.e., in addition to the images themselves included in the medical image study needing to be assigned and analyzed). This additional information received and included in the training data can include, for example, study information (e.g., a study description, a number of series or slices, a modality type, a number of prior studies, a total number of images in prior studies, an imaging protocol used, or the like), patient information (e.g., demographic information, disease history, etc.), one or more prior image studies, one or more exam reports (e.g., a report for the image study, reports for prior image studies, findings, impressions, annotations, pathology reports, etc.), CAD or other AI findings in the image study or prior image studies, reading time information for the image study, an RVU assigned to the image study, or a combination thereof. Accordingly, rather than simply training a model to estimate a difficulty metric for an image study based on a set of image studies and associated labels, which may not represent all factors that make one image study more complex or difficult to analyze than another image study, embodiments described herein train the model using additional relevant information, such as, for example, patient demographic information and medical history information. In particular, a number of prior image studies associated with a patient can impact the difficulty in analyzing a new image study for the patient. For example, when a patient has only a single prior image study available, analyzing a new image study for this patient may be less complex than when the patient has multiple prior image studies available. If a model is not trained with information regarding prior image studies (e.g., numbers, types, findings, progressions, etc.), a model cannot take this factor into account. Thus, by incorporating prior image studies in the training data, embodiments described herein provide more accurate difficulty metrics than other systems, which results in a more balanced distribution and workload and more accurate medical reports and findings. For example, by including prior image studies in the training data, the models described herein may use the number of prior image studies, the types of prior image studies, a timing of prior image studies, findings in prior image studies (e.g., how a lesion or area of interest has changed over time between one or more prior studies), or the like to output an improved difficulty metric for an unlabeled image study that results in improved user efficiency as well as computing resource efficiency (e.g., given a more accurate initially-assigned metric and associated radiologist assignment).

For example, to assign a difficulty metric to an unlabeled medical image study associated with a patient (referred to herein as a “current patient” to distinguish from other patients associated with training data and labeled medical image studies) some embodiments described herein receive prior image study information of the current patient, prior exam information of the current patient, and current exam information of the unlabeled medical image study. The prior image study information may include a number of prior image studies associated with the current patient, a number of image series in a prior image study associated with the current patient, and a total number of images in the prior image studies associated with the current patient. The prior exam information may include findings and impressions in prior exam reports associated with the current patient, and the current exam information may include computer-aided diagnosis (CAD) results of the unlabeled medical image study. The difficulty model 145 uses this information in combination with the unlabeled medical images study itself (and optionally additional information as described above) to estimate a difficulty metric for the unlabeled medical image study. In particular, the difficult model 145 can be trained using training data that includes similar data (prior image study information, prior exam information, and CAD results) for labeled medical image studies to correlate prior image study, exam reports, and CAD results to associated difficulty metrics and, thus, recognize the fact that higher number of prior image studies the more work generally needed to review an image study, especially if there are multiple findings or impressions that could be correlated with CAD results (including AI-driven results) in the image study being assigned a difficulty metric.

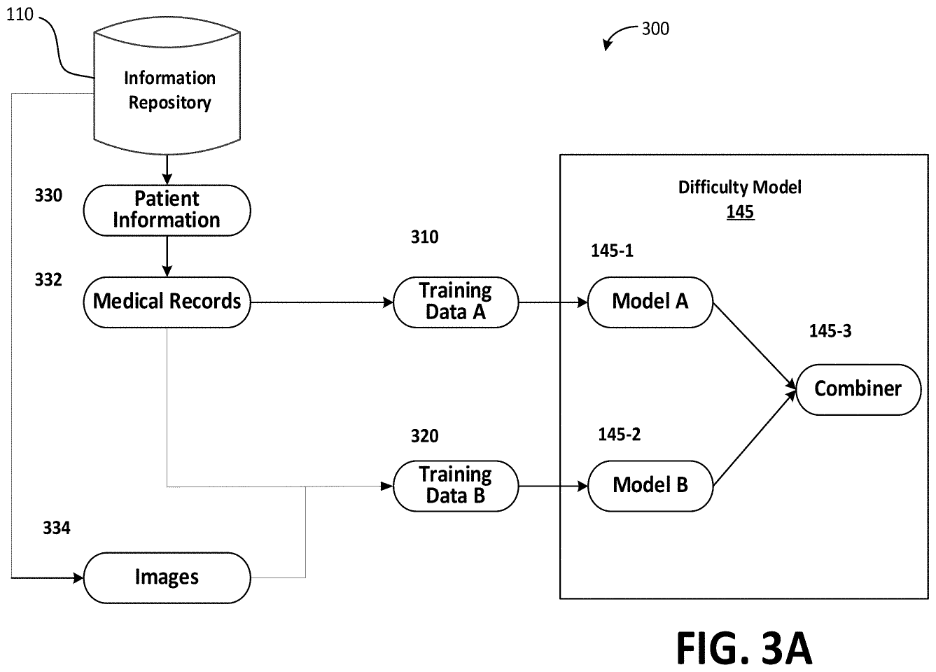

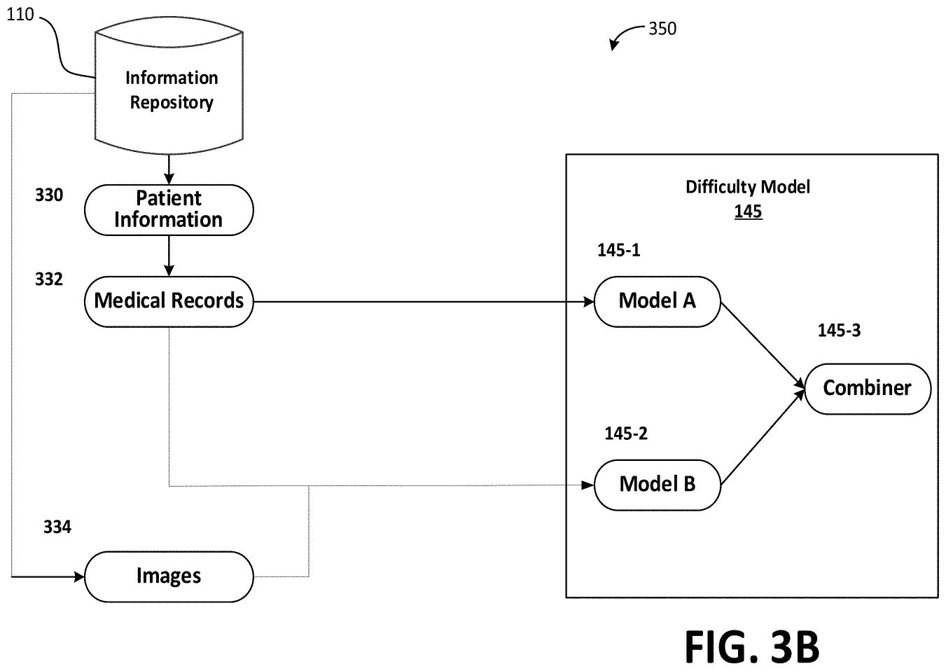

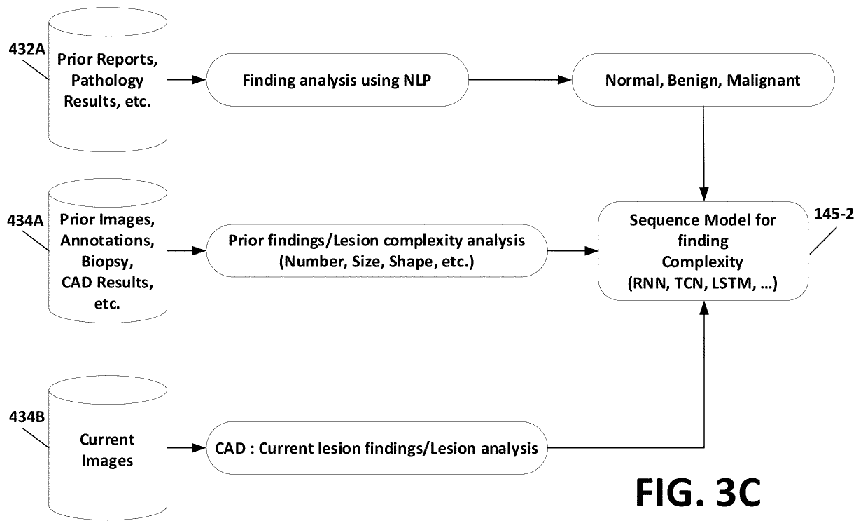

As shown in FIG. 3A, the difficulty model 145 is trained to estimate or output a difficulty metric of a medical image study based on the training data created from a plurality of completed or labeled medical image studies. In an example embodiment, FIG. 3A illustrates a training workflow 300 of the difficulty model 145. As shown in FIG. 3A, in some implementations, the difficulty model 145 includes a model A 145-1, a model B 145-2, and a combiner 145-3. As described in more detail below, each of the models A 145-1 and B 145-2 can be configured to output a difficulty metric (also referred to as a “difficulty sub-metric” herein) and the combiner 145-3 can be configured to generate the overall difficulty metric for a medical image study based on the outputs of the two models A 145-1 and 5 145-2.

For example, in some embodiments, the difficulty model 145 receives training data A 310 and training data B 320 to train the model A 145-1 and the model B 145-2, respectively, wherein each model, once trained, is configured to output a respective difficulty metric (e.g., a difficult sub-metric) for a medical image study. The combiner 145-3 is configured to generate a respective difficulty metric for the medical image study based on the output of the model A 145-1 and the model B 145-2 (e.g., combining the sub-metrics, averaging the sub-metrics, or the like). As illustrated in FIG. 3A, the models 145-1 and 145-2 may be trained using different sets of data. For example, the training data A 310 may include the patient information 330 and the medical records 332 that correspond to a plurality of labeled medical image studies. This patient and procedure information can be pulled from data stored in or available through the information repository 110 using natural language processing (NLP) techniques. For example, NLP techniques can be used to pull and standardize (e.g., categorize) relevant information (e.g., a normal, benign, or malignant finding) from image study reports stored in a RIS.

The patient information 330 may include, for example, demographic information such as a gender, age, weight, medical condition, ethnicity, geographic location, or the like, or a combination thereof. Additionally, the patient information 330 may include, for example, disease history, such as abnormal condition of a part, organ, or system of a patient resulting from various causes, such as infection, inflammation, environmental factors, or genetic defects.

The medical records 332 may include, for example, medical records such as prior reports, findings/impressions, annotations, pathology reports, pathology results, computer aided diagnosis (CAD) or other artificial intelligence (AI) findings in current and prior exams. Additionally, the medical records 332 may include, for example, medical image study descriptions that identify the purpose of the study, type of data collected, and/or how the collected data will be used.

In contrast, the training data B 320 includes the patient information 330, the medical records 332, and images 334 that correspond to the plurality of labeled medical image studies of the information repository 110. The images 334 may include, for example, a study description, number of series images/slices, modality, number of priors, imaging protocol, image volume, relative pathological findings from prior reports images, annotations, biopsies, etc. Additionally, the images 334 may include metadata, such as lesion findings and findings of lesion complexity (e.g., number, size, shape, mass, calcification, etc.) of current and prior images of the images 334.

The model A 145-1 may be, for example, a machine learning model for estimating relationships between a dependent variable (e.g., difficulty metric, procedure information, etc.) and one or more independent variables, such as the patient information 330 and the medical records 332. In some embodiments, the model A 145-1 is configured to use regression analysis using the patient and procedure information for the labeled image studies. For example, the difficulty model 145 utilizes the model A 145-1 to identify causal relationships between a dependent variable and a collection of independent variables in a fixed dataset, such as medical study information (e.g., the patient information 330 and the medical records 332) of a medical image study.

The model B 145-2 may be, for example, a sequence machine learning model for estimating an output (e.g., difficulty metric, medical image study complexity, etc.) based on a sequence of data inputs, such as the patient information 330, the medical records 332, and the images 334. The model B 145-2 may be, for example, a recurrent neural network (RNN), temporal convolutional network (TCN), long-short term memory (LSTM), or any other machine learning model capable of analyzing time-series data. For example, the difficulty model 145 utilizes the model B 145-2 to identify causal relationships between a complexity of a medical image study (e.g., output, difficulty metric, etc.) and time series data (e.g., the patient information 330, the medical records 332, and the images 334) corresponding to a patient of the medical image study.

As noted above, the combiner 145-3 is configured to generate a final or overall difficulty metric for a medical image study based on the output of the model A 145-1 and the model B 145-2. For example, the combiner 145-3 may, for example, determine a sum (e.g., difficulty metric) of respective outputs of the model A 145-1 and the model B 145-2. In another example, the combiner 145-3 may, for example, determine an average (e.g., difficulty metric) of respective outputs of the model A 145-1 and the model B 145-2. Other pooling, stacking, and boosting algorithms can be used by the combiner 145-3 in various embodiments.

While FIG. 3A illustrates the patient information 330, the medical records 332, and images 334 as separate inputs of the information repository 110, in some embodiments, the server 105 may receive the inputs in various combinations and from various sources. Accordingly, the patient information 330, the medical records 332, and images 334 are shown as separate inputs in FIG. 3A for illustrative purposes.

After the difficulty model 145 (i.e., the models 145-1 and 145-2) are trained using the labeled image studies and associated information, the model 145 can be used to assign a difficulty metric to an unlabeled image study needing review. For example, FIG. 3B illustrates a medical image study workflow 350 for assigning a difficulty metric to an unlabeled medical image study using the difficulty model 145. As shown in FIG. 3B, the difficulty model 145 receives an unlabeled medical image study and associated information, which may include, for example, a patient information 430, medical records 432, and images 434 included in the unlabeled image study. As illustrated in FIG. 3B, the patient information 430, the medical records 432 are input into the model A 145-1, and the patient information 330, the medical records 332, and the images 334 are input into the model B 145-2. The outputs of the model A 145-1 and the model B 145-2 are combined via the combiner 145-3 to generate a difficulty metric for the unlabeled medical image study.

While FIG. 3B illustrates the patient information 430, the medical records 432, and images 434 as separate inputs of the information repository 110, in some embodiments, the server 105 may receive the inputs in various combinations and from various sources. Accordingly, the patient information 430, the medical records 432, and images 434 are shown as separate inputs in FIG. 3B for illustrative purposes.

In an example embodiment, FIG. 3C illustrates a scoring workflow 355 of a model of the difficulty model of FIG. 3B for assigning a difficulty value to an unlabeled medical image study using the model B 145-2 of the difficulty model 145. As shown in FIG. 3C, the difficulty model B 145-2 receives an unlabeled medical image study and associated information, which may include, for example, a medical records 432A, prior images 434A, and current images 434B included in the unlabeled image study. As illustrated in FIG. 3C, relevant information of prior reports of the medical records 432A such as, for example, a normal, benign, or malignant finding are input into the model B 145-2. Additionally, prior findings related to lesion complexity of prior images, annotations, biopsies, or computer aided diagnosis (CAD) results of the prior images 434A such as, for example, number of lesions, size of lesions, lesion mass, calcification, etc., are input into the model B 145-2. Also, relevant information of the current images 434B such as, for example, current lesion findings, CAD results, etc., are input into the model B 145-2. In this example embodiment, the model B 145-2 outputs a difficulty value that corresponds to identified causal relationships complexity of findings of the unlabeled medical image study. For example, a higher number of lesions findings in current images of an unlabeled medical image study with respect to prior reports can result in a higher difficulty value.

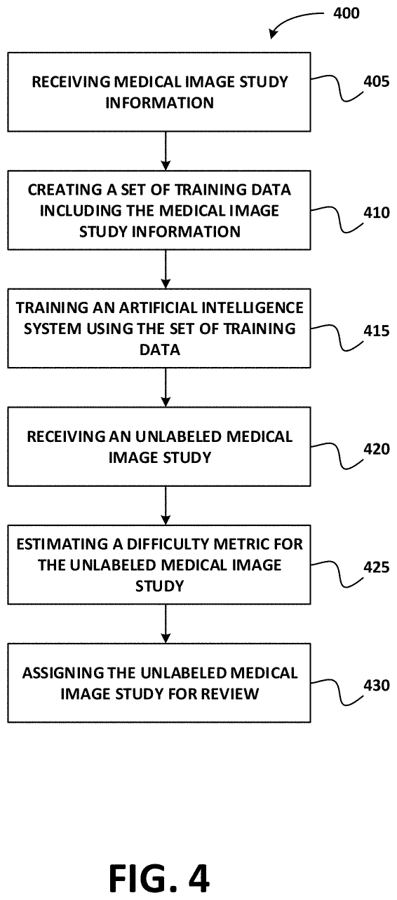

The difficulty metric assigned to the medical image study needing review can be used to assign the medical image study to a healthcare provider (e.g., a radiologist) and, in particular, can be used to provide a more balanced distribution of image studies needing review. For example, FIG. 4 is a flowchart illustrating a method 400 for estimating a difficulty metric of a medical image study and assigning the medical image study for review based on the difficulty metric. The method 400 may be performed by the server 105 (i.e., the electronic processor 130 implementing the difficulty model 145). However, in other embodiments, the method 400 may be performed by multiple servers or systems in various configurations and distributions. The method 400 includes receiving labeled medical image study information including labeled medical image studies (image studies with an associated difficult metric) and associated information as described above (at block 405). For example, labeled medical image studies may be uploaded to the information repository 110, and the server 105 may receive the medical image studies and use the medical image studies to access or receive the associated information regarding the medical image studies from (e.g., through a push or pull configuration) the information repository 110, other data sources, or a combination thereof as described above. In particular, as noted above, the labeled medical image study information can include not only labeled image studies but also associated patient and procedure information, reports, and prior image studies and associated reports and findings.

The method 400 includes creating a set of training data including the labeled medical image study information (at block 410). For example, the server 105 may utilize a plurality of received medical image studies uploaded to the information repository 110 to create a labeled set of data that may include information, such as input-output pairs, in memory 135. The input-output pairs may include a set of features of a medical image study (e.g., input) and difficulty metric corresponding to the set features (e.g., output). As noted above, the labels (i.e., the difficulty metrics) may be defined manually by an expert or determined based on reading information.

The method 400 includes training an artificial intelligence system using the set of training data (at block 415). For example, the server 105 inputs a created labeled set of data into the difficulty model 145. In some embodiments, the server 105 reserves a segment of the plurality of received medical image studies uploaded to the information repository 110 to create a test set of data, which qualifies performance of the difficulty model 145. For example, as training the difficulty model 145 with an initial set of data, the server 105 inputs the test set of data into the difficulty model 145 to determine an accuracy of the difficulty model 145. In some embodiments, the server 105 may iteratively input labeled set of data and the test set of data into the difficulty model 145 until performance of the difficulty model 145 reaches a target accuracy. As also noted above, in some embodiments, the difficulty model 145 includes multiple (e.g., two) models, wherein each model can be trained using a particular subset of the training data.

The method 400 also includes, after training the difficulty model 145, receiving an unlabeled medical image study (at block 420). For example, an unlabeled medical image study may be uploaded to the information repository 110. In this example, the server 105 can use information included in the uploaded image study to access or receive associated medical information regarding the unlabeled medical image study, such as from the information repository 110, other data sources, or a combination thereof. Again, as noted above, the associated information can include patient information, procedure information, prior image studies, reports associated with prior image studies, pathology reports, CAD or AI results for the prior image studies, the unlabeled image study, or combinations thereof. It should be understood that the type of information used to estimate a difficulty metric for an unlabeled image study via the difficulty model 145 is similar to the data used to train the difficulty model 145 (e.g., the same type of data with the exception of a label).

As illustrated in FIG. 4, the server 105 provides the medical study information of the unlabeled medical image study to the difficulty model 145, which estimates a difficulty metric for the unlabeled medical image study (at block 425).

The method 400 further includes assigning the unlabeled medical image study for review based on the estimated difficulty metric (at block 430). For example, the server 105 assigns an unlabeled medical image study to an identifier of a care provider in a worklist table stored in the memory 135. In this example, the server 105 may assign the unlabeled medical image study to a care provider with an available status based on the worklist table. In another example, the server 105 receives a total workload (e.g., a cumulative difficulty metric for a care provider) from a worklist table stored in the memory 135 for each care provider working within the system 100. In this example, the server 105 may assign the unlabeled medical image study to a care provider using the total workload for each care provider and a determined difficulty metric for the unlabeled medical image study. Also, the server 105 may assign the unlabeled medical image study to adhere to a set of parameters, such as a cumulative difficulty metric threshold, an average total workload of care providers of the worklist table, etc. to balance workloads. In some embodiments, the method 400 includes transmitting a received unlabeled medical image study to a workstation of a care provider. For example, the processor 130 may route the unlabeled medical image study to workstation 120 of a care provider using updated information (e.g., assignment information) of a worklist table stored in the memory 135.

Accordingly, embodiments described herein account for the many factors that can contribute to the difficulty of reviewing a medical image study, including whether a current study has multiple prior studies and prior findings or reports and patient information. Using artificial intelligence allows embodiments described herein to learn patterns of study difficulty taking into account these factors, which allows for more accurate difficulty metrics and, consequently, more balanced workload distribution among radiologists.

Various features and advantages of the embodiments are set forth in the following claims.

What is claimed is:

1. A computer-implemented method for assigning a medical image study for review, the method comprising:
receiving a plurality of labeled medical image studies, each of the plurality of labeled medical image studies including a medical image study and a label representing a difficulty of the respective medical image study;
receiving, for each of the plurality of labeled medical image studies, one or more prior image studies of a patient associated with the respective labeled medical image study;
creating a set of training data including the plurality of labeled medical image studies and the one or more prior image studies received for each of the plurality of labeled medical image studies;
training an artificial intelligence (AI) system using the set of training data;
receiving prior image study information of a current patient associated with an unlabeled medical image study, wherein the prior image study information includes a number of prior image studies associated with the current patient, a number of image series in a prior image study associated with the current patient, and a total number of images in the prior image studies associated with the current patient;
receiving prior exam information of the current patient, wherein the prior exam information includes findings and impressions in prior exam reports associated with the current patient;
receiving current exam information of the unlabeled medical image study, wherein the current exam information includes computer-aided diagnosis (CAD) results of the unlabeled medical image study;
estimating, using the AI system as trained, a difficulty metric for the unlabeled medical image study based on the unlabeled medical image study, the prior image study information of the current patient, the prior exam information of the current patient, and the current exam information of the unlabeled medical image study; and
assigning the unlabeled medical image study for review based on the difficulty metric.

2. The method of claim 1, wherein training the AI system using the set of training data comprises:
training a first machine learning model of the AI system using a first set of training data, the first set of training data including, for each of the plurality of labeled medical image studies, information regarding the patient associated with the respective labeled medical image study and information regarding a procedure associated with the respective labeled medical image study; and
training a second machine learning model of the AI system using a second set of training data, the second set of training data including, for each of the plurality of labeled medical image studies, the information regarding the patient associated with the respective labeled medical image study, the information regarding the procedure associated with the respective labeled medical image, images associated with the respective labeled medical image study, and images associated with the one or more prior image studies received for the respective labeled medical image study.

3. The method of claim 2, wherein at least one of the first set of training data and the second set of training data includes information regarding a pathology report associated with the one or more prior image studies received for each of the plurality of labeled medical image studies.

4. The method of claim 2, wherein estimating the difficulty metric for the unlabeled medical image study using the AI system comprises:
generating a first difficulty sub-metric for the unlabeled medical image study using the first machine learning model;
generating a second difficulty sub-metric for the unlabeled medical image study using the second machine learning model; and
generating the difficulty metric for the unlabeled medical image study based on the first difficulty sub-metric and the second difficulty sub-metric.

5. The method of claim 2, wherein the first machine learning model of the AI system uses regression analysis and wherein the second machine learning model of the AI system uses sequence modeling.

6. The method of claim 1, further comprising, receiving, for each of the plurality of labeled medical image studies, information regarding the patient associated with the respective labeled medical image study, wherein creating the set of training data includes creating the set of training data including the plurality of labeled medical image studies, the one or more prior image studies received for each of the plurality of labeled medical image studies, and the information regarding the patient received for each of the plurality of labeled medical image studies.

7. The method of claim 1, wherein the label of each of the plurality of labeled medical image studies is based on a read time of the respective labeled medical image study or an assigned value received from an expert.

8. The method of claim 1, further comprising, standardizing the findings and impressions in the prior exam reports using natural language processing (NLP).

9. A system for assigning a medical image study for review, the method comprising:
an electronic processor configured to:
receive a plurality of labeled medical image studies, each of the plurality of labeled medical image studies including a medical image study and a label representing a difficulty of the respective medical image study;
receive, for each of the plurality of labeled medical image studies, one or more prior image studies of a patient associated with the respective labeled medical image study;
create a set of training data including the plurality of labeled medical image studies and the one or more prior image studies received for each of the plurality of labeled medical image studies;
train an artificial intelligence (AI) system using the set of training data;
receive prior image study information of a current patient associated with an unlabeled medical image study, wherein the prior image study information includes a number of prior image studies associated with the current patient, a number of image series in a prior image study associated with the current patient, and a total number of images in the prior image studies associated with the current patient;
receive prior exam information of the current patient, wherein the prior exam information includes findings and impressions in prior exam reports associated with the current patient;
receive current exam information of the unlabeled medical image study, wherein the current exam information includes computer-aided diagnosis (CAD) results of the unlabeled medical image study;
estimate, using the AI system as trained, a difficulty metric for the unlabeled medical image study based on the unlabeled medical image study, the prior image study information of the current patient, the prior exam information of the current patient, and the current exam information of the unlabeled medical image study; and
assign the unlabeled medical image study for review based on the difficulty metric.

10. The system of claim 9, wherein the electronic processor is configured to training the AI system using the set of training data by:
training a first machine learning model of the AI system using a first set of training data, the first set of training data including, for each of the plurality of labeled medical image studies, information regarding the patient associated with the respective labeled medical image study and information regarding a procedure associated with the respective labeled medical image study; and
training a second machine learning model of the AI system using a second set of training data, the second set of training data including, for each of the plurality of labeled medical image studies, the information regarding the patient associated with the respective labeled medical image study, the information regarding the procedure associated with the respective labeled medical image, images included in the respective labeled medical image, and images included in the one or more prior image studies received for the respective labeled medical image.

11. The system of claim 10, wherein at least one of the first set of training data and the second set of training data includes information regarding a pathology report associated with the one or more prior image studies received for each of the plurality of labeled medical image studies.

12. The system of claim 10, wherein the electronic processor is configured to estimate the difficulty metric for the unlabeled medical image study using the AI system by:
generating a first difficulty sub-metric for the unlabeled medical image study using the first machine learning model;
generating a second difficulty sub-metric for the unlabeled medical image study using the second machine learning model; and
generating the difficulty metric for the unlabeled medical image study based on the first difficulty metric and the second difficulty metric.

13. The system of claim 10, wherein the first machine learning model of the AI system uses regression analysis and wherein the second machine learning model of the AI system uses sequence modeling.

14. The system of claim 10, wherein the electronic processor is further configured to receive, for each of the plurality of labeled medical image studies, information regarding the patient associated with the respective labeled medical image study, wherein the set of training data includes the plurality of labeled medical image studies, the one or more prior image studies received for each of the plurality of labeled medical image studies, and the information regarding the patient received for each of the plurality of labeled medical image studies.

15. The system of claim 10, wherein the label of each of the plurality of labeled medical image studies is based on a read time of the respective labeled medical image study or an assigned value received from an expert.

16. The system of claim 10, further comprising, standardizing the findings and impressions in the prior exam reports using natural language processing (NLP).

17. Non-transitory computer-readable medium storing instructions that, when executed by an electronic processor, perform a set of functions, the set of functions comprising:
receiving a plurality of labeled medical image studies, each of the plurality of labeled medical image studies including a medical image study and a label representing a difficult of the respective medical image study;
receiving, for each of the plurality of labeled medical image studies, one or more prior image studies of a patient associated with the respective labeled medical image study;
creating a set of training data including the plurality of labeled medical image studies and the one or more prior image studies received for each of the plurality of labeled medical image studies;
training an artificial intelligence (AI) system using the set of training data;
receiving prior image study information of a current patient associated with an unlabeled medical image study, wherein the prior image study information includes a number of prior image studies associated with the current patient, a number of image series in a prior image study associated with the current patient, and a total number of images in prior image studies associated with the current patient;
receiving prior exam information of the current patient, wherein the prior exam information includes findings and impressions in prior exam reports associated with the current patient;
receiving current exam information of the unlabeled medical image study, wherein the current exam information includes computer-aided diagnosis (CAD) results of the unlabeled medical image study;
estimating, using the AI system as trained, a difficulty metric for the unlabeled medical image study based on the unlabeled medical image study, the prior image study information of the current patient, the prior exam information of the current patient, and the current exam information of the unlabeled medical image study; and
assigning the unlabeled medical image study for review based on the difficulty metric.

18. The non-transitory computer-readable medium of claim 17, wherein training the AI system using the set of training data includes:
training a first machine learning model of the AI system using a first set of training data, the first set of training data including, for each of the plurality of labeled medical image studies, information regarding the patient associated with the respective labeled medical image study and information regarding a procedure associated with the respective labeled medical image study; and
training a second machine learning model of the AI system using a second set of training data, the second set of training data including, for each of the plurality of labeled medical image studies, the information regarding the patient associated with the respective labeled medical image study, the information regarding the procedure associated with the respective labeled medical image, image associated with the respective labeled medical image study, and images associated with the one or more prior image studies received for the respective labeled medical image study.

19. The non-transitory computer-readable medium of claim 18, wherein estimating the difficulty metric for the unlabeled medical image study using the AI system includes:
generating a first difficulty sub-metric for the unlabeled medical image study using the first machine learning model;
generating a second difficulty sub-metric for the unlabeled medical image study using the second machine learning model; and
generating the difficulty metric for the unlabeled medical image study based on the first difficulty sub-metric and the second difficulty sub-metric.

20. The non-transitory computer-readable medium of claim 17, wherein the label of each of the plurality of labeled medical image studies is based on a read time of the respective labeled medical image study.
